(19) United States
(12) Patent Application Publication
(10) Pub. No.: US 2024/0050336 A1
(43) Pub. Date: Feb. 15, 2024

(54) AEROSOL DEVICE CONTAINING A COMPOSITION COMPRISING A SOLID FATTY ALCOHOL AND A LIQUEFIED GAS

(71) Applicant: L'Oreal, Paris (FR)

(72) Inventors: Guillaume RONCHARD, SAINT-OUEN (FR); Sophie BOUXIROT, SAINT-OUEN (FR); Anne-Laure FAMEAU, SAINT-OUEN (FR)

(21) Appl. No.: 18/267,714

(22) PCT Filed: Dec. 17, 2021
(86) PCT No.: PCT/EP2021/086638

§ 371 Date: Jun. 15, 2023

(30) Foreign Application Priority Data
Dec. 18, 2020 (FR) FR2013746

(51) Int. Cl. A61K 8/34; A61Q 5/12; A61Q 5/06; A61K 8/04

(52) U.S. Cl. CPC A61K 8/342; A61Q 5/12; A61Q 5/06; A61K 8/046; A61K 2800/87; A61K 2800/31

(57) ABSTRACT

The present invention relates to an aerosol device containing a composition, notably a cosmetic hair composition, comprising at least 2% by weight of one or more fatty alcohols including at least one solid fatty alcohol, optionally one or more C₂-C₆ monoalcohols, at least 30% of a propellant chosen from liquefied gases and optionally one or more liquid fatty substances other than liquid fatty alcohols. The invention also relates to the use of the sprayed composition according to the invention for shaping and/or conditioning keratin fibres, and notably for defining the hair curls.

The present invention relates to an aerosol device containing a composition, notably a cosmetic composition, notably a hair composition, comprising at least 2% by weight of one or more fatty alcohols including at least one solid fatty alcohol, optionally one or more C₂-C₆ monoalcohols, at least 30% of a propellant chosen from liquefied gases and optionally one or more liquid fatty substances other than liquid fatty alcohols.

The invention also relates to a process for shaping and/or conditioning keratin fibres, in particular human keratin fibres such as the hair, comprising at least one step of applying the composition according to the invention to said fibres.

The invention lastly relates to the use of the composition according to the invention sprayed from the aerosol device for shaping and/or conditioning keratin fibres, in particular human keratin fibres such as the hair, and notably for providing softness, a smooth, non-tacky feel and sheen, for facilitating the disentangling of the hair, for defining the hair curls, and for providing control of the frizziness and volume of the head of hair. The aerosol format makes it possible to deliver a light mist that does not weigh the hair down and that is easy to spread on the hair.

Many people are unsatisfied with the appearance of their hair: in particular people who have curly hair may wish to maintain the curls but to obtain better curl definition and better regularity, better movement and less volume and frizziness, even in humid conditions. Furthermore, some people find it difficult to control, define and/or style their curly hair.

Some people also wish for their hair to be soft and shiny.

There is a need to provide compositions that can simplify the styling routine, in particular in the case of people with curly hair.

In particular, there is a need to provide compositions which can maintain hair curls while at the same time giving them good definition and regularity, and which make it possible to obtain good control of the frizziness and volume of the hair, which withstands humidity, while at the same time providing haircare and sheen, which may be persistent after shampooing several times.

In point of fact, the Applicant has just discovered, surprisingly, that an aerosol device comprising a composition comprising a combination of one or more solubilized fatty alcohols and at least 30% of a liquefied gas, makes it possible to solve the abovementioned problems.

One subject of the present invention is thus an aerosol device comprising a composition comprising:
a) at least 2% by weight, relative to the total weight of the composition, of one or more fatty alcohols, the composition comprising at least one or more solid fatty alcohols and at least one or more liquid fatty alcohols,
b) at least 30% by weight, relative to the total weight of the composition, of one or more propellants chosen from liquefied gases, and
c) optionally one or more C₂-C₆ monoalcohols,
d) optionally one or more liquid fatty substances other than liquid fatty alcohols.
   Advantageously, the fatty alcohol(s) are solubilized in the composition.

The Applicant has notably observed that the composition according to the invention makes it possible to obtain well-defined curls, good control of the frizziness and volume of the hair, and to give the hair good manageability. In addition, the composition according to the invention affords a hair conditioning effect and makes it possible in particular to give a pleasant cosmetic feel, notably a smooth, non-tacky, non-greasy and non-waxy feel, to facilitate the disentangling of the hair and to provide sheen. The composition does not give a greasy effect and affords volume control. The use of an aerosol device enables good distribution of the composition on the head of hair.

In addition, the compositions according to the invention are advantageously clear or transparent, hence an original visual effect for hair products of this type in transparent containers.

Furthermore, the composition according to the invention spreads easily on the hair, notably on dry hair, in order to obtain conditioning and/or styling care.

A subject of the invention is also a process for shaping and/or conditioning keratin fibres, in particular human keratin fibres such as the hair, comprising at least one step of applying to said fibres a composition sprayed from an aerosol device according to the invention.

Other subjects, characteristics, aspects and advantages of the invention will emerge even more clearly on reading the description and the example that follows.

In the present description, the expression “at least one” is equivalent to the expression “one or more” and can be replaced therewith; the term “between” is equivalent to the expression “ranging from” and can be replaced therewith, and implies that the limits are included.

The term “keratin fibres” means fibres of human or animal origin, such as head hair, bodily hair, the eyelashes, the eyebrows, wool, angora, cashmere or fur. According to the present invention, the keratin fibres are preferably human keratin fibres, more preferentially the hair.

The composition according to the invention is advantageously in the form of a clear to translucent fluid, more preferentially a clear fluid. This property is notably advantageous in the case of transparent containers, in particular containers made of PET (polyethylene terephthalate).

The clarity of the composition according to the invention may be characterized by measuring its turbidity, by turbidimetry (in NTU units). In the context of the present invention, the turbidity measurements were performed with a turbidimeter, model HI 88713-ISO from the company Hanna Instruments.

Preferably, the turbidity of the compositions according to the invention, measured at room temperature (25° C.) and atmospheric pressure, is less than 400 NTU units, more preferentially between 1 and 250 NTU units, better still between 3 and 200 NTU units, more particularly from 3 to 50 NTU units.

Fatty Alcohols

The composition according to the invention comprises at least 2% by weight of one or more fatty alcohols relative to the total weight of the composition, at least one of which is solid.

The term “fatty alcohol” means a linear or branched, saturated or unsaturated, non-glycerolated and non-oxyalkylenated aliphatic alcohol comprising from 6 to 40 carbon atoms and comprising at least one hydroxyl group. The fatty alcohols preferably contain from 8 to 30 carbon atoms, preferably from 12 to 24 carbon atoms and better still from 14 to 22 carbon atoms.

According to the invention, the fatty alcohols are preferably solubilized in the composition.

The term “solubilized fatty alcohol” means that the fatty alcohols do not precipitate from the composition and/or that they do not form crystals. When the composition is checked visually with the naked eye, it is homogeneous, single-phase, and clear or translucent.

The term “liquid” refers to a compound that is liquid at a temperature of 25° C. and at atmospheric pressure (760 mmHg; i.e. 1.013×10⁵ Pa). Preferably, this compound has a viscosity of less than or equal to 2 Pa·s, preferably less than or equal to 1 Pa·s, more preferentially less than or equal to 0.1 Pa·s measured at a temperature of 25° C. and at a shear rate of 1 s⁻¹.

The term “solid” refers to a compound that is not liquid at a temperature of 30° C. and at atmospheric pressure (760 mmHg; i.e. 1.013×10⁵ Pa).

Solid Fatty Alcohols

The solid (non-liquid) fatty alcohols according to the invention are chosen from saturated or unsaturated, linear or branched alcohols including from 8 to 30 carbon atoms, preferably from 12 to 24 carbon atoms, better still from 14 to 22 carbon atoms.

More preferentially, the solid (non-liquid) fatty alcohols according to the invention are chosen from saturated linear alcohols including from 8 to 30 carbon atoms, preferably from 12 to 24 carbon atoms, better still from 14 to 22 carbon atoms.

Preferably, the solid fatty alcohols according to the invention are chosen from fatty alcohols having a melting point above 30° C., preferentially above 35° C.

Preferably, mention may be made, for example, of cetyl alcohol, stearyl alcohol, behenyl alcohol and myristyl alcohol, and mixtures thereof.

Liquid Fatty Alcohols

The liquid fatty alcohols of the invention may be saturated or unsaturated.

The saturated liquid fatty alcohols are preferably branched. They may optionally comprise in their structure at least one aromatic or non-aromatic ring. Preferably, they are acyclic.

More particularly, the saturated liquid fatty alcohols of the invention are chosen from 2-octyldodecanol, isostearyl alcohol, 2-hexyldecanol, 2-butyloctanol and 2-undecylpentadecanol. Octyldodecanol is most particularly preferred.

The liquid unsaturated fatty alcohols contain in their structure at least one double or triple bond.

Preferably, they bear in their structure one or more double bonds. When several double bonds are present, there are preferably 2 or 3 of them, and they may be conjugated or unconjugated.

The liquid unsaturated fatty alcohols may be linear or branched. They may optionally comprise in their structure at least one aromatic or non-aromatic ring. Preferably, they are acyclic.

More particularly, the liquid unsaturated fatty alcohols of the invention may be chosen from oleyl alcohol, linoleyl alcohol, linolenyl alcohol and undecylenyl alcohol, and mixtures thereof. Oleyl alcohol is particularly preferred.

Preferably, the composition according to the invention comprises at least one solid fatty alcohol and at least one liquid fatty alcohol.

According to this embodiment, the hair conditioning properties are improved, notably a smoother natural feel, and the hair is softer and more shiny. The hair does not have a greasy/waxy appearance.

Preferably, the ratio of the weight concentrations of solid fatty alcohol(s)/total fatty alcohol(s) is greater than 0.25 and preferably ranges from 0.3 to 1, preferentially from 0.3 to 0.75, more particularly from 0.4 to 0.70.

The composition according to the invention may comprise a total amount of one or more solid fatty alcohols ranging from 0.5% to 25% by weight, preferably from 1% to 15% by weight, and more preferentially from 1.5% to 10% by weight, relative to the total weight of the composition.

The composition according to the invention may comprise a total amount of one or more liquid fatty alcohols ranging from 0.5% to 20% by weight, preferably from 1% to 10% by weight, and more preferentially from 1.5% to 8% by weight, relative to the total weight of the composition.

The composition according to the invention may comprise a total amount of one or more fatty alcohols ranging from 2% to 25% by weight, preferably from 2.5% to 20% by weight, more preferentially from 3% to 15% by weight and better still from 3% to 12% by weight, relative to the total weight of the composition.

Propellants

The aerosol device according to the invention comprises a composition comprising one or more propellants chosen from liquefied gases.

As examples of liquefied gases, mention may be made of dimethyl ether, chlorinated and/or fluorinated hydrocarbons, such as trichlorofluoromethane, dichlorodifluoromethane, chlorodifluoromethane, 1,1,1,2-tetrafluoroethane, chloropentafluoroethane, 1-chloro-1,1-difluoroethane or 1,1-difluoroethane, or volatile hydrocarbons, notably such as C₃-C₅ alkanes, such as propane, isopropane, n-butane, isobutane or pentane, alone or as a mixture. Preferably, the hydrocarbons are chosen from propane, isopropane, n-butane and isobutane, alone or as a mixture.

According to a particular embodiment, the propellant comprises one or more liquefied gases, preferably chosen from dimethyl ether and C₃-C₅ alkanes and in particular propane, n-butane or isobutane, alone or as a mixture, more preferentially chosen from C₃-C₅ alkanes and in particular n-butane or isobutane, alone or as a mixture.

According to the invention, the propellant(s) are present in the composition comprising the other ingredients, or alternatively are present in a compartment separate from the compartment comprising the other ingredients of the composition, within the aerosol device.

The propellant(s) are generally present in a total amount ranging from 30% to 95% by weight, preferably from 40% to 90% by weight, more preferentially from 50% to 90% by weight and better still from 60% to 90% by weight, relative to the total weight of the composition.

C₂-C₆ Monoalcohols

The composition according to the present invention may comprise one or more C₂-C₆ monoalcohols.

The C₂-C₆ monoalcohols are notably ethanol, isopropanol, tert-butanol or n-butanol. Preferably, the alcohol used is ethanol.

Preferably, the composition according to the invention comprises one or more C₂-C₆ monoalcohols.

Preferably, when it is (they are) present, the C₂-C₆ alcohol(s) are present notably in a total amount ranging from 0.5% to 68%, preferably from 3% to 60% by weight, in particular ranging from 7% to 50% by weight, or even from 8% to 30% by weight, relative to the total weight of the composition.

Liquid Fatty Substances

The composition according to the present invention may comprise one or more liquid fatty substances other than liquid fatty alcohols.

The term “fatty substance” means an organic compound that is insoluble in water at ordinary room temperature (25° C.) and at atmospheric pressure (760 mmHg, i.e. 1.013×10⁵ Pa), with a solubility in water of less than 5% by weight, preferably less than 1% by weight and even more preferentially less than 0.1% by weight. They have in their structure at least one hydrocarbon-based chain including at least 6 carbon atoms. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane. The fatty substances of the invention are not etherified with oxyalkylenated or glycerolated groups. Preferably, they do not contain in their structure any carboxylic acid functions COOH.

The term “hydrocarbon” means a compound comprising solely carbon and hydrogen atoms.

The term “fatty acid” means a compound comprising at least one linear or branched, saturated or unsaturated carboxylic acid function, comprising from 6 to 40 carbon atoms.

The term “fatty ester” means an ester of a fatty acid and/or of a fatty alcohol.

The term “fatty ether” means a compound comprising at least one linear or branched, saturated or unsaturated ether function, comprising at least one hydrocarbon-based group containing from 6 to 40 carbon atoms.

The term “non-silicone” means a compound whose structure does not comprise any silicon atoms, and which therefore notably does not comprise any siloxane groups.

More particularly, the liquid fatty substances are non-silicone substances.

In particular, the fatty substances of the invention are not (poly)oxyalkylenated or (poly)glycerolated ethers.

The term “oil” means a “fatty substance” that is liquid at room temperature (25° C.) and at atmospheric pressure (760 mmHg or 1.013×10⁵ Pa).

More particularly, the fatty substances are chosen from C₆-C₁₆ liquid hydrocarbons, liquid hydrocarbons comprising more than 16 carbon atoms, non-silicone oils of animal origin, oils of triglyceride type of plant or synthetic origin, fluoro oils, liquid esters of a fatty acid and/or of a fatty alcohol other than triglycerides, liquid fatty ethers, dialkyl carbonates, and mixtures thereof.

It is recalled that the fatty esters and acids more particularly contain at least one saturated or unsaturated, linear or branched hydrocarbon-based group, comprising from 6 to 40 and better still from 8 to 30 carbon atoms, which is optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ liquid hydrocarbons, they may be linear or branched, optionally cyclic, and are preferably alkanes. Examples that may be mentioned include hexane, cyclohexane, undecane, dodecane, tridecane or isoparaffins, such as isohexadecane, isodecane or isododecane, and mixtures thereof. C₆-C₁₆ isoparaffins are particularly preferred, more preferentially isododecane.

The linear or branched hydrocarbons of mineral or synthetic origin comprising more than 16 carbon atoms are preferably chosen from liquid paraffins, petroleum jelly, liquid petroleum jelly, polydecenes and hydrogenated polyisobutene such as Parleam®, and mixtures thereof.

The triglyceride oils of plant or synthetic origin are preferably chosen from liquid triglycerides of fatty acids including from 6 to 30 carbon atoms, such as heptanoic or octanoic acid triglycerides, or else, for example, sunflower oil, linseed oil, olive oil, safflower oil, argan oil, maize germ oil, wheat germ oil, soybean oil, marrow seed oil, grapeseed oil, sesame oil, hazelnut oil, apricot kernel oil, macadamia oil, arara oil, castor oil, avocado oil, caprylic/capric acid triglycerides, for instance those sold by the company Stéarinerie Dubois or those sold under the names Miglyol® 810, 812 and 818 by the company Dynamit Nobel, shea butter oil, and mixtures thereof.

Fatty Esters

As regards the liquid esters of fatty acids and/or of fatty alcohols other than the triglycerides mentioned previously and the fatty esters a) described previously, mention may notably be made of esters of saturated or unsaturated, linear C₁-C₂₆ or branched C₃-C₂₆ aliphatic monoacids or polyacids and of saturated or unsaturated, linear C₁-C₂₆ or branched C₃-C₂₆ aliphatic monoalcohols or polyalcohols, the total carbon number of the esters being greater than or equal to 10.

Preferably, for the esters of monoalcohols, one at least from among the alcohol or of the acid from which the esters of the invention are derived is branched and/or unsaturated.

Among the monoesters, mention may be made of dihydroabietyl behenate; octyldodecyl behenate; isocetyl behenate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl palmitate, isopropyl palmitate, 2-ethylhexyl palmitate, 2-octyldecyl palmitate, alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of isopropyl palmitates, alkyl myristates such as isopropyl myristate, isocetyl stearate, 2-ethylhexyl isononanoate, isodecyl neopentanoate, isononyl isononanoate, and mixtures thereof. Still within the context of this variant, use may also be made of esters of C₄-C₂₂ dicarboxylic or tricarboxylic acids and of C₁-C₂₂ alcohols, and esters of monocarboxylic, dicarboxylic or tricarboxylic acids and of C₂-C₂₆ dihydroxy, trihydroxy, tetrahydroxy or pentahydroxy alcohols.

Mention may notably be made of: diethyl sebacate; diisopropyl sebacate; diisopropyl adipate; di-n-propyl adipate; dioctyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; glyceryl trilactate; glyceryl trioctanoate; propylene glycol dioctanoate; neopentyl glycol diheptanoate; diethylene glycol diisononanoate; and mixtures thereof.

The composition may also comprise, as fatty ester, sugar esters and diesters of C₆-C₃₀ and preferably C₁₂-C₂₂ fatty acids. It is recalled that the term “sugar” means oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may notably be chosen from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, notably, oleopalmitate, oleostearate and palmitostearate mixed esters.

More particularly, use is made of monoesters and diesters and notably sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by the company Amerchol, which is a methylglucose dioleate.

According to a preferred embodiment, use may also be made of dialkyl carbonates, in particular di(C₁-C₁₂)alkyl carbonates, preferably di(C₄-C₁₂)alkyl carbonates notably such as dicaprylyl carbonate.

Fatty Ethers

The liquid fatty ethers may be chosen from dialkyl ethers, in particular di(C₁-C₁₂)alkyl ethers, preferably di(C₄-C₁₂)alkyl ethers, notably such as dicaprylyl carbonate.

Preferably, the composition according to the invention comprises one or more liquid fatty substances other than liquid fatty alcohols.

According to one embodiment, the liquid fatty substance(s) are chosen from C₆-C₁₆ alkanes, in particular branched C₈-C₁₆ alkanes, fatty ethers, dialkyl carbonates and mixtures thereof.

According to a preferred embodiment, the liquid fatty substance(s) are chosen from branched C₈-C₁₆ alkanes, di(C₁-C₁₂)alkyl ethers, di(C₁-C₁₂)alkyl carbonates and mixtures thereof, more preferentially from branched C₈-C₁₆ alkanes, di(C₁-C₁₂)alkyl ethers and mixtures thereof.

The composition according to the invention may comprise the liquid fatty substance(s) other than liquid fatty alcohols in a total content ranging from 0.5% to 40% by weight, preferably from 1% to 25% by weight, preferentially from 1.5% to 20% by weight, relative to the total weight of the composition.

The composition according to the invention may be for cosmetic use. For the purposes of the invention, the term “cosmetic” means a composition of pleasant appearance, odour and feel. Preferably, the composition is for topical application to the skin and keratin fibres, and more especially to the scalp, the hair and the eyelashes, and better still to the hair.

The composition according to the invention may be in the form of an alcoholic or aqueous-alcoholic solution of liquid or semi-liquid consistency.

When the composition according to the invention comprises water, the water content of the composition according to the invention is advantageously less than 10% by weight, preferably ranges from 1% to 8% by weight, in particular from 1% to 5% by weight, relative to the total weight of the composition.

Preferably, the composition according to the invention is anhydrous.

For the purposes of the present invention, the term “anhydrous” means that the water content is less than 1% by weight, preferably less than 0.5% by weight relative to the total weight of the composition, and better still the composition is free of water.

In particular, the composition according to the invention does not comprise any water added during its preparation, the residual water possibly being provided by the starting materials used for its preparation.

The composition according to the invention may also comprise one or more additional organic solvents, other than the C₂-C₆ monoalcohols and liquid fatty substances described previously, for example chosen from polyols, notably those containing from 2 to 6 carbon atoms, for instance glycerol, propylene glycol, butylene glycol, pentylene glycol, hexylene glycol, dipropylene glycol or diethylene glycol; and polyol ethers such as 2-butoxyethanol, propylene glycol monomethyl ether, and diethylene glycol monoethyl ether and monomethyl ether; and mixtures thereof.

Preferably, the composition according to the invention comprises less than 10% by weight, preferentially less than 5%, more preferentially less than 2% by weight and better still less than 1% by weight of polyol(s), relative to the total weight of the composition. More particularly, the composition does not comprise any polyol.

The composition according to the invention may comprise one or more surfactants chosen from nonionic, anionic, amphoteric and cationic surfactants, and mixtures thereof.

The composition according to the invention may also comprise one or more additional compounds chosen from natural or synthetic thickeners or viscosity regulators; UV-screening agents, fillers such as nacres, titanium dioxide, resins and clays, fragrances, peptizers, vitamins, acidic agents, alkaline agents, dyestuffs, nacreous agents, opacifiers, film-forming polymers, fixing polymers, conditioning agents, solid fatty substances other than solid fatty alcohols, antioxidants and preserving agents.

A person skilled in the art will take care to select the optional additional compounds and the amount thereof such that they do not harm the properties of the composition of the present invention, notably the solubilization of the fatty alcohols.

These additives may be present in the composition according to the invention in an amount ranging from 0 to 20% by weight, relative to the total weight of the composition.

When the composition is aqueous, the pH of the composition according to the invention generally ranges from 2 to 12, preferably from 2.5 to 9, preferentially from 3 to 8 and better still from 4 to 7.

The pH of the composition may be adjusted to the desired value by means of the basifying agents or acidifying agents that are customarily used. Among the basifying agents, examples that may be mentioned include aqueous ammonia, alkanolamines, and mineral or organic hydroxides. Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

The cosmetic composition may be thus rinsed off or left on after having been applied to the keratin materials, for example rinsed off with water, after an optional leave-on time. The composition is preferably a leave-on composition.

Aerosol Device

The aerosol device according to the present invention comprising the composition is constituted of a container containing said composition and of a means for spraying said composition.

Preferably, the aerosol device according to the invention makes it possible to spray said composition in spray or mousse form, preferably in spray form.

The composition of use according to the invention is packaged under pressure, in an aerosol device, for example a monobloc device, which comprises a spraying means and a container.

The spraying means is generally constituted of a dispensing valve controlled by a dispensing head, itself comprising a nozzle through which the composition of the invention is sprayed. The valve may comprise an additional gas intake.

The container containing the pressurized composition may be opaque or transparent. It may be made of glass, polymer or metal, and may optionally be coated with a protective varnish coat.

A subject of the present invention is also a process for shaping and/or conditioning keratin fibres, in particular human keratin fibres such as the hair, comprising at least one step of applying to said fibres a composition as described previously.

This or these steps of applying the composition according to the invention may optionally be preceded by at least one step of washing said fibres using a shampoo.

Moreover, this or these steps of applying the composition according to the invention may optionally be followed by a step of leaving the composition on, for example a leave-on time of 5 seconds to 15 minutes, notably 15 seconds to 5 minutes; then followed by an optional rinsing step, for example with water; and/or a drying step.

Preferably, the keratin fibres are not rinsed after the step(s) of applying the composition according to the invention. The expression “are not rinsed” means that no step of rinsing the keratin fibres is performed within a period of less than 30 minutes, preferentially 20 minutes, after the step of applying the composition according to the invention.

Preferably, the composition as described previously is used on curly hair, to define the curls.

The examples that follow serve to illustrate the invention without, however, being limiting in nature.

### EXAMPLES

### Example 1

Compositions A1, B1 and C₁ according to the invention were prepared from the following ingredients, the contents of which are indicated in the tables below (as g of active material).

TABLE 1

| | A1 | B1 | C1 |
|---|---|---|---|
| Ingredients | (invention) | (invention) | (invention) |
| Cetylstearyl alcohol (50/50 by | 9 | 1.93 | 3.86 |
| weight) | | | |
| Oleyl alcohol | 6 | 1.29 | 2.57 |
| Dicaprylyl ether | 8 | 1.71 | 3.43 |
| Butane | 30 | 85 | 70 |
| Ethanol | qs 100 | qs 100 | qs 100 |

The fluid was produced by cold solubilization of the fatty alcohols and dicaprylyl ether in ethanol. The fluid was then pressurized with the liquefied gas in a PET container with a long-skirted valve (Gi: 2×0.5 mm; Ri 0.8 mm); and an additional 0.4 mm gas intake equipped with a V06.261 nozzle borsalino push button 3.9 mm in diameter.

Before pressurization of the composition (without propellant), it was observed that the solid fatty alcohols are solubilized in the compositions according to the invention. No fatty alcohol crystals were observed. The compositions were transparent. The compositions were stable over time. 0.055 g of composition per g of hair was sprayed onto locks of slightly wavy Caucasian hair (curl type 1) from the aerosol device.

Trained experts then evaluated the following properties:

TABLE 2

| | A1 | B1 | C1 |
|---|---|---|---|
| Ease of application to dry hair | +++ | +++ | +++ |
| No greasy feel | ++++ | ++++ | ++++ |
| Volume control | +++ | +++ | +++ |
| Manageability | ++ | +++ | +++ |
| Lightness | ++++ | ++++ | ++++ |
| Sheen | +++ | +++ | +++ |

The application in the form of an aerosol spray enabled homogeneous distribution.

It was observed that the hair treated with these compositions according to the invention was lightweight, smooth to the touch, soft, shiny and had good hairstyling properties (curl definition, frizz control and volume control). The feel was neither greasy nor loaded.

### Example 2

The compositions according to the invention were prepared from the following ingredients, the contents of which are indicated in the tables below (as g of active material).

TABLE 3

| Ingredients | A2 | B2 |
|---|---|---|
| Cetylstearyl alcohol (50/50 by | 1.93 | 5.5 |
| weight) | | |
| Oleyl alcohol | 1.29 | 5.5 |
| Dicaprylyl ether | 1.71 | 10 |
| Ethanol | — | 38.6 |
| Fragrance | 0.45 | 0.4 |
| Butane | 94.4 | — |
| Hydrofluorocarbon 152A | | 40 |

The compositions were transparent. The compositions were stable over time. The application in the form of an aerosol spray enabled homogeneous distribution on the head of hair.

It was observed that the hair treated with these compositions according to the invention was lightweight, smooth to the touch, soft, shiny and had good hairstyling properties (curl definition, frizz control and volume control). The feel was neither greasy nor loaded.

### Example 3

The following compositions were prepared from the ingredients of which the contents are indicated in the table below (an g of active material).

TABLE 4

| | A | B | C |
|---|---|---|---|
| | invention | comparative | comparative |
| Cetylstearyl alcohol (solid fatty | 3.86 | — | 6.43 |
| alcohol) | | | |
| Oleyl alcohol (liquid | 2.57 | 6.43 | — |
| fatty alcohol) | | | |
| Dicaprylyl ether | 3.43 | 3.43 | 3.43 |
| Butane | 70 | 70 | 70 |
| Ethanol | qs 100 | qs 100 | qs 100 |

The fluid was produced by cold solubilization of the fatty alcohols and dicaprylyl ether in ethanol. The fluid was then pressurized with the liquefied gas in a PET container with a long-skirted valve (Gi: 2×0.5 mm; Ri 0.8 mm); and an additional 0.4 mm gas intake equipped with a V06.261 nozzle borsalino push button 3.9 mm in diameter.

The compositions were transparent and stable over time.

Protocol 0.055 g of composition per g of hair was sprayed onto locks of slightly wavy Caucasian hair (curl type 1) from the aerosol device.

The feel quality (non-greasy, non-waxy) and the transfer quality were then evaluated by six trained experts, on dry hair, in a “blind” test, on a scale going from 0 (very greasy, very waxy) to 10 (very sparingly greasy, very sparingly waxy) for the quality of the feel and from 0 (no transfer) to 10 (substantial transfer).

The Evaluation is by Touch:

The expert takes the lock between thumb and index finger, descends from the top of the lock to the ends and at the same time effects an oscillating movement with the thumb to get a good feel for the presence of any deposit.

At the same time, the expert evaluates the presence/sensation of transfer of product onto the fingers.

Quality of the Feel

| | A | B | C |
|---|---|---|---|
| | invention | comparative | comparative |
| Mean | 6.5 | 3.9 | 4 |

Transfer

| | A | B | C |
|---|---|---|---|
| | invention | comparative | comparative |
| Mean | 3.0 | 5.7 | 6.7 |

Composition A according to the invention leads to a better feel (less greasy, less waxy) and made it possible to reduce the transfer in comparison with compositions B and C.

1-18. (canceled)

19. A composition comprising:
a) at least one fatty alcohol comprising at least one solid fatty alcohol and at least one liquid fatty alcohol in a total amount of at least 2% by weight, relative to the total weight of the composition,
b) at least one liquified gas propellent in an amount of at least 30% by weight, relative to the total weight of the composition,
c) optionally at least one C₂-C₆ monoalcohol, and
d) optionally at least one liquid fatty substance other than the at least one liquid fatty alcohol.

20. The composition of claim 19, wherein the at least one solid fatty alcohol is chosen from cetyl alcohol, stearyl alcohol, behenyl alcohol, myristyl alcohol, or mixtures of two or more thereof.

21. The composition of claim 19, wherein the at least one liquid fatty alcohol is chosen from 2-octyldodecanol, isostearyl alcohol, 2-hexyldecanol, 2-butyloctanol, 2-undecylpentadecanol, oleyl alcohol, linoleyl alcohol, linolenyl alcohol, undecylenyl alcohol, or mixtures of two or more thereof.

22. The composition of claim 19, wherein the total amount of solid fatty alcohol(s) ranges from 0.5% to 25% by weight, relative to the total weight of the composition.

23. The composition of claim 19, wherein the total amount of liquid fatty alcohol(s) ranges from 0.5% to 20% by weight, relative to the total weight of the composition.

24. The composition of claim 19, wherein the weight ratio of the total amount of solid fatty alcohol(s) to the total amount of fatty alcohol(s) is greater than 0.25:1.

25. The composition of claim 19, wherein the weight ratio of the total amount of solid fatty alcohol(s) to the total amount of fatty alcohol(s) ranges from 0.4:1 to 0.7:1.

26. The composition of claim 19, wherein the total amount of fatty alcohol(s) ranges from 2% to 25% by weight, relative to the total weight of the composition.

27. The composition of claim 19, wherein the total amount of fatty alcohol(s) ranges from 3% to 15% by weight, relative to the total weight of the composition.

28. The composition of claim 19, wherein the at least one fatty alcohol is soluble in the composition.

29. The composition of claim 19, wherein the at least one liquified gas propellant is chosen from dimethyl ether, chlorinated and/or fluorinated hydrocarbons, volatile hydrocarbons, or mixtures of two or more thereof.

30. The composition of claim 19, wherein the total amount of liquified gas propellant(s) ranges from 30% to 95% by weight, relative to the total weight of the composition.

31. The composition of claim 19, wherein the composition comprises at least one C₂-C₆ monoalcohol chosen from ethanol, isopropanol, tert-butanol, n-butanol, or mixtures of two or more thereof.

32. The composition of claim 19, wherein the total amount of C₂-C₆ alcohol(s) ranges from 0.5% to 68% by weight, relative to the total weight of the composition.

33. The composition of claim 19, wherein the at least one liquid fatty substance other than the at least one liquid fatty alcohol is chosen from C₆-C₁₆ alkanes, fatty ethers, dialkyl carbonates, or mixtures of two or more thereof.

34. The composition of claim 19, wherein the total amount of the at least one liquid fatty substance other than the at least one liquid fatty alcohol ranges from 0.5% to 40% by weight, relative to the total weight of the composition.

35. The composition of claim 19, wherein the composition further comprises at least one polyol in a total amount of less than 10% by weight, relative to the total weight of the composition.

36. The composition of claim 19, wherein the composition is anhydrous.

37. An apparatus for spraying a composition onto keratin fibers, the composition comprising:
a) at least one fatty alcohol comprising at least one solid fatty alcohol and at least one liquid fatty alcohol in a total amount of at least 2% by weight, relative to the total weight of the composition,
b) at least one liquified gas propellent in an amount of at least 30% by weight, relative to the total weight of the composition,
c) optionally at least one C₂-C₆ monoalcohol, and
d) optionally at least one liquid fatty substance other than the at least one liquid fatty alcohol.

38. A method for shaping and/or conditioning keratin fibers, comprising applying to the keratin fibers a composition comprising:
a) at least one fatty alcohol comprising at least one solid fatty alcohol and at least one liquid fatty alcohol in a total amount of at least 2% by weight, relative to the total weight of the composition,
b) at least one liquified gas propellent in an amount of at least 30% by weight, relative to the total weight of the composition,
c) optionally at least one C₂-C₆ monoalcohol, and
d) optionally at least one liquid fatty substance other than the at least one liquid fatty alcohol.
